(19) United States
(12) Patent Application Publication
(10) Pub. No.: US 2018/0140520 A1
(43) Pub. Date: May 24, 2018

(54) ORAL CARE DEVICE

(71) Applicant: Conopco, Inc., d/b/a UNILEVER, Englewood Cliffs, NJ (US)

(72) Inventors: Brian Joseph GROVES, Port Sunlight, Wirral, Merseyside (GB); Adam John LIMER, Northwich, Cheshire (GB); Carole Jane PHILPOTTS, Upton, Wirral, Merseyside (GB); William John WILSON, Bebington, Wirral, Merseyside (GB)

(73) Assignee: Conopco, Inc., d/b/a UNILEVER, Englewood Cliffs, NJ (US)

(21) Appl. No.: 15/572,949

(22) PCT Filed: May 5, 2016
(86) PCT No.: PCT/EP2016/060126

§ 371 Date: Nov. 9, 2017

(30) Foreign Application Priority Data
Jun. 5, 2015 (EP) 15170829.4

(51) Int. Cl. A61K 8/29; A61Q 11/00; A61K 8/24; A61K 8/25; A61C 19/06

(52) U.S. Cl. CPC A61K 8/29; A61Q 11/00; A61K 2800/87; A61K 8/25; A61C 19/066; A61K 8/24

(57) ABSTRACT

An oral care kit comprising: i) an application device ii) first non-aqueous composition comprising a whitening component and a water insoluble and/or slightly soluble calcium source; and (iii) a second composition comprising a phosphate source in which the second composition is stored separately from the first composition prior to usage of the product; and in which the first and second compositions are adapted to be applied sequentially to the application device, the device then being placed on the surface 15 of the teeth for sustained contact.

### FIELD OF THE INVENTION

The invention relates to an oral care products for the remineralisation and whitening of teeth.

### BACKGROUND OF THE INVENTION

Teeth are important both functionally and aesthetically. There is a need for strong healthy teeth that appear white and glossy.

Teeth whitening strips are seen as a convenient way of achieving white teeth.

Whitening strips in which the active whitening agent is hydrogen peroxide are disclosed in U.S. Pat. No. 5,879,691 and U.S. Pat. No. 6,893,629.

However, there remains the need for a simple and effective way whiten and strengthen the teeth.

### SUMMARY OF THE INVENTION

Accordingly the present invention relates to an oral care kit comprising:

i) an application device suitable for being placing on the surface of the teeth for sustained contact;

ii) a first non-aqueous composition comprising a whitening component and a water insoluble and/or slightly soluble calcium source; and

(iii) a second composition comprising a phosphate source in which the second composition is stored separately from the first composition prior to usage of the product; and in which the first and second compositions are adapted to be applied sequentially to the application device.

The invention further relates to a method of cosmetically whitening the teeth comprising the following steps:

i) selecting an application device;

ii) applying a first non-aqueous composition comprising a whitening component and a water insoluble and/or slightly soluble calcium source to a surface of the application device;

iii) followed by applying a second composition comprising a phosphate source to the same surface of the application device as the first composition was applied to;

iv) placing the device iii) on the teeth for sustained contact.

### DETAILED DESCRIPTION OF THE INVENTION

The delivery device of the invention is preferably flexible, more preferably the device comprises a strip of an orally acceptable flexible material. The surface of the strip is capable of being applied to a tooth surface.

Preferably the device has an elongate shape of a length sufficient that when placed against the front surface of a user's teeth it extends across a plurality of teeth, and of sufficient width that it extends at least from the gumline of the teeth to the crowns of the teeth. The elongate shape is such that it minimises the need for subsequent applications and time to cover all the user's teeth.

In a further embodiment the strip is such that it can be sufficient that when placed against the front surface of a user's teeth it extends across a plurality of teeth, and of sufficient width that it extends at least from the front gumline of the teeth to the crowns of the teeth and to the gumline behind the users teeth leading to total coverage of the teeth above the gumline.

Preferably the application device is rectangular in shape.

It is preferable if the device, preferably a strip is formed from an absorbent material, more preferably the device (preferably strip) is a fabric formed from a water soluble, water insoluble, or water hydratable polymer or other material, such as other polymers (e.g., polypropylene, polyethylene, etc.) and cellulose, most preferably the natural fibre is a cotton mix.

If a strip it is preferable if the application strip is greater than 0.001 mm thick and preferably less than 2.5 mm thick.

First compositions of the invention comprise water insoluble and/or slightly soluble calcium source. Soluble and insoluble calcium source, as used herein, refers to the solubility of the calcium source in water. Soluble means a source that dissolves in water to give a solution with a concentration of at least 0.1 moles per litre at room temperature. Insoluble means a source that dissolves in water to give a solution with a concentration of less than 0.001 moles per litre at room temperature. Slightly soluble, therefore, is defined to mean a source that dissolves in water to give a solution with a concentration of greater than 0.001 moles per litre at room temperature and less than 0.1 moles per litre at room temperature. Substantially free of, as used herein, means less than 1.5%, and preferably, less than 1.0%, and most preferably, from 0.0 to 0.75% by weight, based on total weight of the oral care composition, including all ranges subsumed therein. The calcium source suitable for use in this invention is limited only to the extent that the same may be used in an oral cavity. In a preferred embodiment, the calcium source employed is insoluble or slightly soluble in water, but most preferably, insoluble in water.

Illustrative examples of the types of calcium source that may be used in this invention include, for example, calcium phosphate (i.e., added), calcium gluconate, calcium oxide, calcium lactate, calcium carbonate, calcium hydroxide, calcium sulfate, calcium carboxymethyl cellulose, calcium alginate, calcium salts of citric acid, calcium silicate, mixtures thereof or the like. In a preferred embodiment the calcium source is calcium silicate. In a more preferred embodiment, the calcium silicate used is (CaSᵢO₃) whereby the same is made commercially available under the name Microcal ET by Ineos Silicas, Ltd.

In yet another preferred embodiment, the calcium source is insoluble calcium silicate, present as the composite material calcium oxide-silica (CaO—SiO₂) as described in commonly-owned application Publication No. 2008/015117.

When a calcium silicate composite material is employed, the ratio of calcium to silicon (Ca:Si) may be from 1:10 to 3:1. The Ca:Si ratio is preferably from 1:5 to 2:1, and more preferably, from 1:3 to 2:1, and most preferably, from about 1:2 to 2:1. The calcium silicate may comprise mono-calcium silicate, bi-calcium silicate, or tri-calcium silicate whereby ratios of calcium to silicon (Ca:Si) should be understood to be atom ratios.

The calcium source employed in this invention may be in a crystalline or amorphous state, and preferably, the same is in an amorphous state. In an often preferred embodiment, the calcium source is in a mesoporous state, i.e. the source is a material having pores with diameters from 1 nm to 50 microns. Mesoporous calcium silicate (MCS) is often preferred.

The MCS which may be used in this invention can be made by combining a calcium salt, a silica precursor like silicate and a structure-directing agent to yield a solid suitable for calcinating. A more detailed description of the process that may be conducted to make the MCS suitable for use in this invention is described in the aforementioned commonly-owned application, Publication No. WO 2008/015117.

The amount of calcium source in the composition present as the second layer of this invention is typically from 0.1 to 50%, and preferably, from 1 to 30%, and most preferably, from 5 to 20% by weight of the oral care composition based on total weight of the oral care composition and including all ranges subsumed therein.

The first composition is non-aqueous, that is that the composition comprises less than 1 wt % of the total first composition of water, preferably less than 0.5 wt % of water.

The first composition of the invention comprises a whitening source, preferably titanium dioxide. A preferred form of titanium dioxide is calcium silicate coated TiO2. Examples of preferred forms of calcium silicate coated titanium dioxide are disclosed in WO2012/031786 and WO2012/031785.

Preferably the level of titanium dioxie is 1-30 wt % or more preferably 5-20 wt % of the second layer.

The second composition comprises a phosphate source. The phosphate source that may be used in this invention is limited only to the extent that the same may be used in a composition suitable for use in an oral cavity. Illustrative examples of the types of phosphate source suitable for use in this invention include monosodium phosphate, sodium dihydrogen phosphate, disodium hydrogen phosphate, sodium pyrophosphate, tetrasodium pyrophosphate, sodium tripolyphosphate, sodium hexametaphosphate, potassium dihydrogenphosphate, trisodium phosphate, tripotassium phosphate, mixtures thereof or the like. The phosphate source is preferably one which is water soluble.

Typically, the phosphate source makes up from 0.5 to 15%, and preferably, from 2 to 12%, and most preferably, from 4 to 9% by weight of the composition used in the third layer, based on total weight of the composition of the third layer and including all ranges subsumed therein. In a preferred embodiment, the phosphate source used is one which results in an oral care composition having a pH from 5.5 to 8, preferably from 6 to 7.5, and most preferably, about neutral. In a most preferred embodiment, the phosphate source used is trisodium phosphate and monosodium dihydrogen phosphate at a trisodium phosphate to monosodium dihydrogen phosphate weight ratio of 1:4 to 4:1, preferably 1:3 to 3:1, and most preferably, from 1:2 to 2:1, including all ratios subsumed therein.

It is preferable if the second composition comprises an aqueous base that is that the composition comprises greater than 50 wt % of the serum composition of water, more preferably greater than 70 wt %.

The oral care compositions described herein may comprise ingredients which are common in the art, such as:
antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds such as 2,2′ methylenebis-(4-chloro-6-bromophenol);
anti-inflammatory agents such as ibuprofen, flurbiprofen, aspirin, indomethacin, etc.;
anti-caries agents such as sodium trimetaphosphate and casein;
plaque buffers such as urea, calcium lactate, calcium glycerophosphate and polyacrylates;
vitamins such as Vitamins A, C and E;
plant extracts;
desensitizing agents, e.g. potassium citrate, potassium chloride, potassium tartrate, potassium bicarbonate, potassium oxalate, and potassium nitrate;
anti-calculus agents, e.g. alkali-metal pyrophosphates, hypophosphite-containing polymers, organic phosphonates and phosphocitrates, etc.;
biomolecules, e.g. bacteriocins, antibodies, enzymes, etc.;
flavors, e.g., peppermint and spearmint oils;
proteinaceous materials such as collagen;
preservatives;
opacifying agents;
coloring agents like FD&C blue, yellow and/or red dyes/colorants;
pH-adjusting agents;
sweetening agents;
surfactants, such as anionic, cationic and zwitterionic or amphoteric surfactants (e.g., sodium lauryl sulfate, sodium dodecylbenzene sulfonate);
particulate abrasive materials such as abrasive silicas, aluminas, calcium carbonates, zirconium silicate, polymethylmethacrylate, dicalciumphosphates, calcium pyrophosphates, hydroxyapatites, trimetaphosphates, insoluble hexametaphosphates as well as agglomerated particulate abrasive materials
fluoride sources like sodium fluoride, stannous fluoride, sodium; monofluorophosphate, zinc ammonium fluoride, tin ammonium fluoride, calcium fluoride, cobalt ammonium fluoride or mixtures thereof;
polymeric compounds which can enhance the delivery of active ingredients such as antimicrobial agents can also be included. Examples of such polymers are copolymers of polyvinylmethylether with maleic anhydride and other similar delivery enhancing polymers, e.g., those described in DE-A03,942,643; buffers and salts to buffer the pH and ionic strength of the oral care compositions; and
other optional ingredients that may be included are, e.g., bleaching agents such as peroxy compound, e.g., potassium peroxydiphosphate, effervescing systems such as sodium bicarbonate/citric acid systems, color change systems, and the like.

Such ingredients common in the art typically and collectively make-up less than 20% by weight of the oral care composition, and preferably, from 0.0 to 15% by weight, and most preferably, from about 0.01 to about 12% by weight of the oral care composition, including all ranges subsumed therein.

Suitable carrier humectants are preferably used in the oral care composition of the present invention and they include, for example, glycerin, sorbitol, propylene glycol, dipropylene glycol, diglycerol, triacetin, mineral oil, polyethylene glycol (preferably, PEG-400), alkane diols like butane diol and hexanediol, ethanol, pentylene glycol, or a mixture thereof. The carrier humectants should, in any case, be substantially free of water, and preferably, anhydrous. The same, for example, can be used in solid form, whereby glycerin is the preferred carrier humectant. Such carriers are particularly suitable in compositions used in the second layer.

The carrier humectant is used to take the balance of the compositions up to 100%, and the same may be present in the range of from 10 to 90% by weight of the oral care composition. Preferably, the carrier humectant makes up from 25 to 80%, and most preferably, from 45 to 70% by weight of the oral care composition, based on total weight of the oral care composition and including all ranges subsumed therein.

On the final coated device before application to the teeth it is preferable if the thickness of the first backing layer is greater than 0.001 mm thick and preferably less than 2.5 mm thick, second layer comprising the second composition is less than about 1 mm thick, preferably less than about 0.5 mm thick, and more preferably from about 0.001 to about 0.3 mm thick and third layer (if present) is less than about 1 mm thick, preferably less than about 0.5 mm thick, and more preferably from about 0.001 to about 0.3 mm thick.

In an alternative mode of the invention, the compositions are adsorbed by the substrate and layers are not present.

The composition used in the layers of the invention are prepared by conventional methods of making oral care formulations. Such methods include mixing the ingredients under moderate shear and atmospheric pressure.

### Mode of Use

The invention provides a method of whitening and remineralising teeth. The method comprises the step of applying a first composition described above to a surface of an application device followed by application of the second composition to same surface. The device is placed on the teeth such that the treated surface of the device is in sustained contact with the teeth. In the context of the present invention sustained contact means the product is left on the teeth for 1 to 30 minutes, preferably, about 5 to 10 minutes before being removed.

Preferably the application of the oral care product of the invention is carried out once daily for a period of several consecutive days, in addition to a regular regime of tooth brushing (preferably at least twice daily).

Typically, use (for a period of about two weeks to one month) of the device with the oral care composition of the present invention will result in a new hydroxyapatite layer on teeth that is from 0.5 to 20 microns, and preferably, from 0.75 to 5 microns, including all ranges subsumed therein.

The invention will now be illustrated by the following non-limiting Examples:

### EXAMPLES

A simulated oral fluid was prepared by adding 1.9 L of water to a glass beaker. The following materials were added one by one with continuous stirring, allowing sufficient time for each chemical to fully dissolve before adding the next. Sodium chloride 16.07 g, sodium hydrogen carbonate 0.7 g, potassium chloride 0.448 g, potassium hydrogen phosphate 2.56 g, magnesium chloride hexahydrate 0.622 g, 1M hydrochloric acid 40 ml, calcium chloride 0.1998 g and sodium sulfate 0.1434 g. The pH was adjusted to 7.0 using saturated TRIS buffer and the total volume made up to 2 L using a volumetric flask.

Human extracted incisors and premolars, obtained for research purposes and obtained with informed consent in accordance with the Human Tissue Act were mounted in plastic cuvettes using the following method. The cuvettes were cut to approximately 15 mm height. A commercial fixative (Simplex Rapid) was mixed 2 parts powder to 1 part liquid as described in the manufacturer's instructions. The cuvettes were then completely filled with the resultant solution and left for approximately 10 minutes to allow the fixative to partly set. At this time one tooth root was completely immersed into the fixative, ensuring that the labial side of the tooth was positioned close to the front of the cuvette. Complete setting of the fixative was achieved after 20 minutes. Any exposed dentine is then sealed with clear nail varnish. The teeth are stored in water to prevent dehydration.

A first formulation was prepared by combining the following:

| Ingredient | % w/w |
|---|---|
| Glycerol | 69.9 |
| Calcium Silicate | 20.0 |
| Calcium silicate coated TiO2 | 10.0 |
| Xanthan | 0.1 |
| Total | 100 |

The glycerol and Xanthan were added to the Esco-Labor 1 L mixing vessel and stirring at 65° C. for 30 minutes to disperse the xanthan gum. Once the Xanthan has been fully dispersed the remaining powders are added slowly and mixed to remove any lumps.

A second formulation was prepared by combining the following:

### Formulation and Processing: Activator Solution for the Dropper

| Ingredient | % w/w |
|---|---|
| Water | 95.1 |
| Trisodium phosphate | 1.63 |
| Monosodium phosphate | 1.37 |
| Cellulose Gum | 1.0 |
| Ethylhexyl glycerine | 0.3 |
| Benzyl Alcohol | 0.3 |
| Phenoxy Ethanol | 0.3 |
| Total | 100 |

Water was added to an Esco-Labor 1 L mixing vessel followed by the Sensiva SC50, Ethoxyethanol and Benzyl Alcohol and mixed to disperse. Subsequently SCMC was slowly added to the vessel through the port in the vessel lid and mixed to disperse.

### Example A

A product slurry was prepared by separately mixing the components of first and second formulation 50:50 by weight. 7 g of mixture was applied to a piece of knitted cotton fabric measuring 10×3.5 cm, two samples were prepared. To each sample 5 human teeth were added and the fabric wrapped around the tooth specimens for 30 minutes at 37° C. After the samples were removed from the fabric, rinsed in 40 ml water and mixed gently for 1 minute. Samples were then incubated in simulated oral fluid for 5 hours. The application process was repeated 5 times in total. Colour was measured via chromameter at baseline and after slurry application and incubation. L*a*b* colour parameters were converted to WIO whiteness indices to allow comparison between samples. Colour change is expressed as ΔWIO=WIO(slurry application)−WIO(baseline).

### Example 1

7 g of the second formulation was applied to two 10×3.5 cm piece of knitted cotton fabric and allowed to impregnate the fabric for 4 hours at room temperature. Immediately before use 3.5 g of the first formulation was applied to each fabric sample using a dropping pipette and spread evenly with a spatula. To each sample 5 human teeth were added and the fabric wrapped around the tooth specimens for 30 minutes at 37° C. After the samples were removed from the fabric, rinsed in 40 ml water and mixed gently for 1 minute. Samples were then incubated in simulated oral fluid for 5 hours. The application process was repeated 5 in total. Colour was measured via chromameter at baseline and after slurry application and incubation. L*a*b* colour parameters were converted to WIO whiteness indices to allow comparison between samples. Colour change is expressed as AW10 =WIO(slurry application)−WIO(baseline).

| | 1 Application | 3 Applications | 5 Applications |
|---|---|---|---|
| Example A | 4.75 ± 0.69 | 5.53 ± 1.41 | 16.42 ± 3.31 |
| Example 1 | 6.11 ± 1.59 | 9.36 ± 1.43 | 18.66 ± 3.43 |

The examples demonstrate that sequential application of the composition to the device (strip) and applying the strip to the teeth is more effective than forming a pre-mix of the compositions, applying to the strip and applying the device to the teeth.

1. A method of cosmetically whitening the teeth comprising the following steps:
i) selecting an application device;
ii) applying a non-aqueous composition comprising a phosphate source to the same surface of the application device as the first composition was applied to;
iii) followed by applying a composition comprising a whitening component and a water insoluble and/or slightly soluble calcium source which is calcium silicate to a surface of the application device;
iv) placing the treated surface of device iii) on the teeth for sustained contact.

2. A method according to claim 1 in which the application device is flexible.

3. A method according to claim 1 in which the application device has an elongate shape of a length that when placed against the front surface of a user's teeth extends across a plurality of teeth, and of width that it extends at least from the gumline of the teeth to the crowns of the teeth.

4. A method according to claim 1 in which the application device is substantially rectangular.

5. A method according to claim 1 in which the device is formed from an absorbent material.

6. A method according to claim 1, in which the phosphate source of the composition is a mixture of trisodium phosphate and sodium dihydrogen phosphate.

7. (canceled)

8. A method according to claim 1 in which the whitening source of the composition is titanium dioxide.

9. A method according to claim 1 in which the titanium dioxide is coated with calcium silicate.

10. (canceled)
